SEQUENCE LISTING (41 sequences)

SEQ ID NO: 1 - DNA, 20 bp, Artificial sequence
  CD247zeta-sgRNA-1
gtggaaggcgcttttcaccg

SEQ ID NO: 2 - DNA, 20 bp, Artificial sequence
  CD247zeta-sgRNA-2
aaaggacaagatgaagtgga

SEQ ID NO: 3 - DNA, 20 bp, Artificial sequence
  CD247zeta-sgRNA-3
tttcaccgcggccatcctgc

SEQ ID NO: 4 - DNA, 20 bp, Artificial sequence
  CD247zeta-sgRNA-4
caggcacagttgccgattac

SEQ ID NO: 5 - DNA, 20 bp, Artificial sequence
  CD247zeta-sgRNA-5
gatggaatcctcttcatcta

SEQ ID NO: 6 - DNA, 20 bp, Artificial sequence
  CD247zeta-sgRNA-6
gacgcccccgcgtaccagca

SEQ ID NO: 7 - DNA, 20 bp, Artificial sequence
  CD247zeta-sgRNA-7
ggcacagttgccgattacag

SEQ ID NO: 8 - DNA, 20 bp, Artificial sequence
  CD3epsilon-sgRNA-1
gggcactcactggagagttc

SEQ ID NO: 9 - DNA, 20 bp, Artificial sequence
  CD3epsilon-sgRNA-2
ggcctctgcctcttatcagt

SEQ ID NO: 10 - DNA, 20 bp, Artificial sequence
  CD3epsilon-sgRNA-3
gcctcttatcagttggcgtt

SEQ ID NO: 11 - DNA, 20 bp, Artificial sequence
  CD3epsilon-sgRNA-4
gatgaggatgataaaaacat

SEQ ID NO: 12 - DNA, 20 bp, Artificial sequence
  CD3epsilon-sgRNA-5
gaacttttatctctacctga

SEQ ID NO: 13 - DNA, 20 bp, Artificial sequence
  CD3epsilon-sgRNA-6
ccatataaagtctccatctc

SEQ ID NO: 14 - DNA, 20 bp, Artificial sequence
  CD3epsilon-sgRNA-7
ttgacatgccctcagtatcc

SEQ ID NO: 15 - DNA, 20 bp, Artificial sequence
  CD3epsilon-sgRNA-8
ggggcttcgcggtaaagttg

SEQ ID NO: 16 - DNA, 20 bp, Artificial sequence
  CD3epsilon-sgRNA-9
ggggggcttcgcggtaaagt

SEQ ID NO: 17 - DNA, 20 bp, Artificial sequence
  CDepislon-sgRNA-10
ggctggtgggttgcccccaa

SEQ ID NO: 18 - DNA, 20 bp, Artificial sequence
  CD3epislon-sgRNA-11
gggccccaggccggcggtga

SEQ ID NO: 19 - DNA, 20 bp, Artificial sequence
  CD3gamma-sgRNA-1
actttggcccagtcaatcaa

SEQ ID NO: 20 - DNA, 20 bp, Artificial sequence
  CD3gamma-sgRNA-2
gtgtatgactatcaagaaga

SEQ ID NO: 21 - DNA, 20 bp, Artificial sequence
  CD3gamma-sgRNA-3
tgactatcaagaagatggtt

SEQ ID NO: 22 - DNA, 20 bp, Artificial sequence
  CD3gamma-sgRNA-4
aatatcacatggtttaaaga

SEQ ID NO: 23 - DNA, 20 bp, Artificial sequence
  CD3gamma-sgRNA-5
tttaaagatgggaagatgat

SEQ ID NO: 24 - DNA, 20 bp, Artificial sequence
  CD3gamma-sgRNA-6
gaatctgggaagtaatgcca

SEQ ID NO: 25 - DNA, 20 bp, Artificial sequence
  CD3gamma-sgRNA-7
agtcatacaccttaaccaag

SEQ ID NO: 26 - DNA, 20 bp, Artificial sequence
  CD3gamma-sgRNA-8
ggtggagttcgccagtcgag

SEQ ID NO: 27 - DNA, 20 bp, Artificial sequence
  CD3gamma-sgRNA-9
ggaatgccaaggaccctcga

SEQ ID NO: 28 - DNA, 20 bp, Artificial sequence
  CD3gamma-sgRNA-10
ggactatcaagaagatggtt

SEQ ID NO: 29 - DNA, 20 bp, Artificial sequence
  CD3gamma-sgRNA-11
ggcagttctgacacactgta

SEQ ID NO: 30 - DNA, 20 bp, Artificial sequence
  CD3delta-sgRNA-1
gaacatagcacgtttctctc

SEQ ID NO: 31 - DNA, 20 bp, Artificial sequence
  CD3delta-sgRNA-2
gtttctctctggcctggtac

SEQ ID NO: 32 - DNA, 20 bp, Artificial sequence
  CD3delta-sgRNA-3
gatacctatagaggaacttg

SEQ ID NO: 33 - DNA, 20 bp, Artificial sequence
  CD3delta-sgRNA-4
gctctcagacattacaagac

SEQ ID NO: 34 - DNA, 20 bp, Artificial sequence
  CD3delta-sgRNA-5
ggacctgggaaaacgcatcc

SEQ ID NO: 35 - DNA, 20 bp, Artificial sequence
  CD3delta-sgRNA-6
ggtccaggatgcgttttccc

SEQ ID NO: 36 - DNA, 20 bp, Artificial sequence
  CD3delta-sgRNA-7
ttcctctataggtatcttga

SEQ ID NO: 37 - DNA, 20 bp, Artificial sequence
  CD3delta-sgRNA-8
ggcgtcgtaggtgtccttgg

SEQ ID NO: 38 - DNA, 20 bp, Artificial sequence
  CD3delta-sgRNA-9
ggatggggggaaagccggta

SEQ ID NO: 39 - DNA, 20 bp, Artificial sequence
  CD3delta-sgRNA-10
gaaggacaagatgaagtgga

SEQ ID NO: 40 - DNA, 20 bp, Artificial sequence
  CD3delta-sgRNA-11
gggtagggccgacgtgtcga

SEQ ID NO: 41 - DNA, 20 bp, Artificial sequence
  TCRalpha-sgRNA-1
agagtctctcagctggtaca

(19) United States
(12) Patent Application Publication
(10) Pub. No.: US 2020/0377878 A1
(43) Pub. Date: Dec. 3, 2020

(54) MODIFIED T CELLS AND USES THEREOF

(71) Applicant: NANJING BIOHENG BIOTECH CO., LTD., Nanjing (CN)

(72) Inventors: Yali ZHOU, Nanjing (CN); Jiangtao REN, Nanjing (CN); Xiaohong HE, Nanjing (CN)

(21) Appl. No.: 16/990,407

(22) Filed: Aug. 11, 2020

Related U.S. Application Data
(63) Continuation of application No. PCT/CN2018/116624, filed on Nov. 21, 2018.

(51) Int. Cl. C12N 15/11; C12N 9/22; A61K 35/17; A61P 35/00

(52) U.S. Cl. CPC C12N 15/11; C12N 9/22; C12N 2310/20; A61P 35/00; A61K 35/17

(57) ABSTRACT

The present invention relates to modified T cells with reduced or abolished TCR/CD3 complex expression, and the methods to produce the same. Also included are pharmaceutical compositions comprising the modified T cell for adoptive therapy and treating a condition, such as cancer, infections or autoimmune diseases.

### FIELD OF THE INVENTION

The present invention relates to modified T cells with reduced or abolished TCR/CD3 complex expression, and the methods to produce the same. Also included are pharmaceutical compositions comprising the modified T cell for adoptive therapy and treating a condition, such as cancer, infections or autoimmune diseases.

### BACKGROUND

T cell plays an important role in controlling tumors and pathogen infections. Adoptive T cell therapy is a novel promising therapeutic approach to restore immune competence. Chimeric antigen receptor (CAR) T cells targeting CD19 has achieved durable remission in patients with B cell leukemia and lymphomas. However, a major obstacle of this T cell therapy is customized manufacture of CAR T cells from each patient.

This patient-specific autologous paradigm is a major limiting factor in the large-scale deployment of CAR technology as it requires either execution by a skilled team, with dedicated access to a Good Manufacturing Practice (GMP)-compliant facility or substantial investment in a centralized processing infrastructure. In addition, delays inherent to the generation of a CAR T product preclude immediate administration, thus compromising favorable outcomes for most critically ill patients. Further, autologous product generation may not be feasible for patients who are profoundly lymphopenic due to previous chemotherapy.

To overcome this obstacle, “off-the-shelf” third party products might serve as a universal donor resource. Before adoptive transfer of allogeneic products, graft versus host disease (GvHD) caused by T cell receptor (TCR) complex on T cell surface must be prevented.

Studies have shown that by disrupting components of TCR (α or β chain) in α/β T cell, the TCR/CD3 complex expression is disrupted, leading to abolished GvHD effect of allogeneic T cells (Laurent et al., 2015; Ren et al., 2016). Successful application of T cells with α or β chain disruption in treating various diseases such as cancers, infections or autoimmune diseases has also been reported. For example, Cellectis reported successful elimination of cancer cells and prevention of GvHD in pediatric acute lymphocytic leukemia (ALL) patients treated with TCRα chain knockout CAR T cells (Waseem et al., 2017). Herein, the inventors found disruption of components of CD3, including the CD3γ chain, CD3δ chain, and CD3ε chain, as well as CD247ζ-chain, other than TCR α or β chain could also disrupt TCR/CD3 complex in T cells, resulting in the abolishment of GvHD effect of α/β T cell. Surprisingly, the inventors further observed that CD3γ, CD3δ, and CD3ε and CD247ζdisruption in CAR T cells enhanced its central memory phenotype and tumor killing capability, especially in comparison to TCR α β chain disrupted CAR T cells.

### DESCRIPTION OF THE INVENTION

Thus, in a first aspect, the present invention relates to a modified T cell, wherein the expression level of TCR/CD3 complex is disrupted by repressing or abolishing the expression of at least one gene selected from CD3γ, CD3δ, CD3ε and CD247ζ. In a further embodiment, the modified T cell according to the present invention further exhibits repressed or abolished expression in TCR α and/or β gene.

The present invention also relates to a pharmaceutical composition comprising the modified T cell according to the present invention. In a further embodiment, said pharmaceutical composition is useful in treating or preventing cancer, infections or autoimmune diseases.

For example, cancers that can be treated with modified T cells include but not limited to acute lymphocytic leukemia (ALL), chronic lymphocytic leukemia (CLL), acute myelogenous leukemia (AML), breast cancer, lung cancer, colorectal cancer, gastric cancer, pancreatic cancer, ovarian cancer, metastatic adenocarcinomas, liver metastases, sarcoma, osteosarcoma, neuroblastoma, melanoma, mesothelioma, glioblastoma, glioma, malignant glioma, hepatocellular, non-small cell lung cancer (NSCLC), ganglioneuroblastoma, brain cancer, renal cancer and prostate cancer. Infectious diseases that can be treated with modified T cells include but not limited to infection caused by virus, bacteria, fungi and parasites. Autoimmune diseases that can be treated with modified T cells include but not limited to type I diabetes, celiac disease, Graves' disease, inflammatory bowel disease, multiple sclerosis, psoriasis, rheumatoid arthritis, Addison's disease, Sjögren's syndrome, Hashimoto's thyroiditis, Myasthenia gravis, Vasculitis, Pernicious anemia and systemic lupus erythematosus.

In a second aspect, the present invention relates to a method of enhancing the central memory phenotype of a T cell, comprising disrupting the expression level of TCR/CD3 complex by repressing or abolishing the expression of at least one gene selected from CD3γ, CD3δ, and CD3ε and CD247ζ in said T cell. In a further embodiment, the method according to the present invention further comprises repressing or abolishing the expression of TCR α and/or β gene.

In a third aspect, the present invention relates to a method of enhancing the tumor killing capability of a T cell, comprising disrupting the expression level of TCR/CD3 complex by repressing or abolishing the expression of at least one gene selected from CD3γ, CD3δ, and CD3ε and CD247ζ in said T cell. In a further embodiment, the method according to the present invention further comprises repressing or abolishing the expression of TCR α and/or β gene.

In a fourth aspect, the present invention relates to a method of abolishing the GvHD effect of a T cell, comprising disrupting the expression level of TCR/CD3 complex by repressing or abolishing the expression of at least one gene selected from CD3γ, CD3δ, and CD3ε and CD247ζ in said T cell. In a further embodiment, the method according to the present invention further comprises repressing or abolishing the expression of TCR α and/or β gene.

In one embodiment, repressing or abolishing the expression of a target gene can be achieved using any techniques in the art, including but not limited to gene mutation, RNA-mediated inhibition, DNA gene editing, RNA editing, base editing and the like.

In one embodiment, the disruption of TCR/CD3 complex according to the present invention is obtained by introducing a gene mutation in at least one gene selected from CD3γ, CD3δ, and CD3ε and CD247ζ that results in repressed or abolished expression of said selected gene(s). Examples of gene mutation include without limitation knock-out mutation, a truncation mutation, a point mutation, a missense mutation, a substitution mutation, a frameshift mutation, an insertion mutation, a duplication mutation, an amplification mutation, a translocation mutation, or an inversion mutation, and any other gene mutation that results in a reduction or inactivation in the corresponding gene activity. Methods of generating at least one mutation in a target gene are well known in the art and include, without limitation, random mutagenesis and screening, site-directed mutagenesis, PCR mutagenesis, insertional mutagenesis, physical mutagenesis, chemical mutagenesis, and irradiation. The mutagenesis, which may be specific or random, may be performed, for example, by use of a suitable physical or chemical mutagenizing agent, use of a suitable oligonucleotide, subjecting the DNA sequence to PCR generated mutagenesis, or any combination thereof. Examples of physical and chemical mutagenizing agents include, without limitation, ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the plant cells or tissues to be mutagenized in the presence of the mutagenizing agent of choice under suitable conditions, and then selecting for mutants exhibiting reduced or no expression of the target gene.

In one embodiment, the disruption of TCR/CD3 complex according to the present invention is obtained by RNA-mediated inhibition of the expression level of at least one target gene selected from CD3γ, CD3δ, and CD3ε and CD247ζ. In particular, said RNA-mediated inhibition of the target gene expression is achieved by introducing into a plant cell a polynucleotide encoding a RNA molecule that is essentially identical or essentially complementary to a transcript sequence of the target gene or fragments thereof, wherein the expression of the polynucleotide results in inhibited expression of the target gene in said plant. A construct comprising a polynucleotide encoding a RNA molecule that is essentially identical or essentially complementary to a transcript sequence of the target gene or fragments thereof, wherein the expression of the construct results in inhibited expression of the target gene in said plant is also encompassed in the scope of the invention.

One skilled in the art is aware that the polynucleotides according to the invention have sequence complementarity that need not be 100 percent, but is at least sufficient to provide a RNA molecule permit hybridization to RNA transcribed from the target gene or DNA of the target gene to form a duplex to permit a gene silencing mechanism. Thus, in embodiments, a polynucleotide fragment is designed to be essentially identical to, or essentially complementary to, a sequence of 18 or more contiguous nucleotides in either the target CD3γ, CD3δ, CD3ε and CD247ζ gene sequence or messenger RNA transcribed from the target gene. By “essentially identical” is meant having 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the target gene or RNA transcribed from the target gene; by “essentially complementary” is meant having 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene. In some embodiments, polynucleotide molecules are designed to have 100 percent sequence identity with or complementarity to one allele or one family member of a given target gene.

Many RNA-mediated inhibition methods are known in the art. Non-limiting examples of RNA molecules used in the RNA-mediated inhibition methods include, but are not limited to, antisense RNAs, miRNAs, siRNAs and long non-coding RNAs. Antisense RNA is a single-stranded RNA that is complementary to a messenger RNA (mRNA) strand transcribed in a cell. When anti sense RNA is expressed in a cell, it binds to a specific messenger RNA molecule and inactivates it. An siRNA is a double-stranded RNA molecule, 20-25 base pairs in length. After separating into single strands and integrating into an active RISC complex, it base-pairs to its target mRNA and induces cleavage of the target mRNA, thereby preventing it from being used as a translation template. A miRNA is a small RNA, typically about 21 nucleotides, that has the ability to modulate the expression of a target gene by binding to mRNA for the target protein, leading to destabilization or translational inhibition of the target protein mRNA, ultimately resulting in reduction of the target protein. Methods for selecting and designing siRNAs and miRNAs for gene inhibition are well known in the art. Long non-coding RNAs (long ncRNA or IncRNA) are non-protein coding transcripts longer than 200 nucleotides (Perkel, BioTechniques, 54 (6):301-304 (2013)). In contrast to many small RNAs which exhibit strong conservation across diverse species, long ncRNAs in general lack strong conservation. Long ncRNAs can be categorized, according to their proximity to protein coding genes in the genome, into five categories; sense, antisense, bidirectional, intronic, and intergenic, and regulate gene expression through a diverse group of mechanisms, such as through gene transcription (e.g., through gene-specific transcription regulation and regulation of basal transcription machinery), post-transcriptional regulation (e.g., through mRNA splicing, translation and siRNA-directed gene regulation) or through epigenetic regulation.

In one embodiment, the disruption of TCR/CD3 complex according to the present invention is obtained by gene editing at least one target gene selected from CD3γ, CD3δ, and CD3ε and CD247ζ involving the use of a nuclease. Non-limiting examples of nucleases include but not limited to meganucleases, zinc finger nucleases (ZFNs), transcription activator-like effector-based nucleases (TALEN), and Cas enzyme used in the clustered regularly interspaced short palindromic repeats (CRISPR/Cas) system. In a preferred embodiment, the modified T cell according to the present application is obtained by a CRISPR/Cas system.

Meganucleases, found commonly in microbial species, have the unique property of having very long recognition sequences (>14 bp) thus making them naturally very specific. However, there is virtually no chance of finding the exact meganuclease required to act on a specific DNA sequence. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting amino acids of the meganuclease to design sequence specific meganucelases in a method named rationally designed meganuclease.

Zinc finger nulceases (ZFNs) recognize target DNA in a modular fashion: each protein consists of at least three zinc finger domains, and a single zinc finger domain interacts with a 3-bp sequence, making them ideal programmable sequence-specific DNA-binding proteins

TALENs emerged as a competitive alternative to ZFNs in 2011. Unlike zinc fingers, each repeat domain in TALE proteins recognizes a single base. Four different repeat domains can be mixed and matched to create new DNA-binding proteins, which can be linked to the FokI domain to create a new class of programmable target DNA nucleases. These molecules enable precise targeting and cutting at a specific genomic locus to generate double-strand breaks (DSBs) followed by non-homologous end joining (NHEJ) or homology-directed repair (HDR)-mediated repair, thereby enabling precise genome editing.

Studies using ZFN and TALEN have led to important scientific discoveries and therapeutic development. In fact, a ZFN-based treatment of HIV that disables the HIV co-receptor C—C chemokine receptor type 5 (CCR5) in human primary T cells is currently in clinical trials and has shown great promise. However, the recognition of the target DNA sequence by these protein-based genome engineering systems is determined by protein sequences. Tedious and complex protein engineering and optimization are therefore required for each specific target DNA sequence and delivering many of these proteins into cells for simultaneous multiplexed genetic manipulation is challenging. Given these difficulties, their use for large-scale genomic manipulation or genetic screens has been limited.

The CRISPR technology originates from type II CRISPR systems. Type II CRISPR systems incorporate sequences from invading DNA between CRISPR repeat sequences that are encoded as arrays within the bacterial host genome. Transcripts from the CRISPR repeat arrays are processed into CRISPR RNAs (crRNAs) (Deltcheva et al., 2011), each containing a variable sequence transcribed from the invading DNA, which is known as the “protospacer” sequence, and part of the CRISPR repeat. Each crRNA hybridizes with a second RNA, which is known as the transactivating CRISPR RNA (tracrRNA) (Deltcheva et al., 2011), and these two RNAs form a complex with the Cas9 DNA endonuclease (Jinek et al., 2012). The protospacer-encoded portion of the crRNA guides Cas9 to complementary target DNA sequences and cleaves the DNA if they are adjacent to short sequences known as protospacer adjacent motifs (PAMs). The type II CRISPR system from Streptococcus pyogenes has been adapted for inducing sequence-specific double-strand breaks (DSBs) and targeted genome editing. In 2012, Jinek et al. first demonstrated that the Cas9 protein from Streptococcus pyogenes (SpCas9) can bind with a tracrRNA-crRNA RNA complex to induce DSBs in vitro at a target DNA sequence by Watson-Crick base pairing between the crRNA and target DNA (Jinek et al., 2012). This study also showed that directing Cas9 to bind and cleave a specific DNA sequence did not require an RNA complex. The process can be simply achieved by using a designed, single guide RNA (sgRNA).

Several studies have reported the successful use of the CRISPR/Cas9 system to disrupt B2m, PD1, CTLA4, CCR5, CXCR4, Lag3 and the like in T cells with efficiency ranging from 7% to over 90% using different protocols. In addition to the native type II CRISPR, a new type V CRISPR has been discovered in recent years. To date, the experimentally tested type V CRISPR systems include the use of the following effector proteins which have been redesignated as Cas12a-e: Cas12a (also known as Cpf1; subtype V-A), Cas12b (also known as C2c1; subtype V-B), Cas12c (also known as C2c3; subtype V-C), Cas12d (also known as CasY; subtype V-D) and Cas12e (also known as CasX; subtype V-E), all of which are evolutionarily distinct from Cas9. Methods to design a CRISPR/Cas9 system comprising specific sgRNAs against the target gene and deliver the same are known in the art. For example, the system can be delivered by transfection with a plasmid that encodes Cas and sgRNA, by non-integrating virus such as adenovirus and adenovirus-associated virus (AAV), by Cas ribonucleoproteins (RNP), or by electroporation.

Gene editing may also be achieved using Argonaute (Ago) proteins. All RNA interference pathways use small single-stranded RNA (ssRNA) molecules that guide proteins of the Argonaute (Ago) family to complementary ssRNA targets: RNA-guided RNA interference. Daan C et al. demonstrates that Ago of the bacterium Thermus thermophilus (TtAgo) acts as a barrier for the uptake and propagation of foreign DNA. Despite structural homology to its eukaryotic counterparts, TtAgo functions in host defence by DNA-guided DNA interference. In 2017 a group in Illinois announced using another Argonaute protein taken from Pyrococcus furiosus (PfAgo) along with guide DNA to edit DNA as artificial restriction enzymes (English et al., 2017).

In one embodiment, the disruption of TCR/CD3 complex according to the present invention is obtained by RNA editing of the transcript of at least one gene selected from CD3γ, CD3δ, and CD3ε and CD247ζ. RNA editing is a posttranscriptional process through which the cellular machineries can make discrete changes to specific nucleoside sequences within a RNA molecule, thereby enhancing the RNA and protein diversity (Gott and Emeson, 2000). RNA editing may involve nucleobase modifications such as cytidine to uridine conversion mediated by a cytidine deaminase or adenosine to inosine conversion involving an Adenosine Deaminases Acting on RNA (ADAR), as well as non-templated nucleotide additions and insertions. It has also been reported that a CRISPR system comprising Cas13a was used for targeted knockdown of endogenous transcripts with comparable levels of knockdown as RNA interference, and improved specificity (Abudayyeh et al., 2017).

In one embodiment, the T cell according to the present application is a T cell, CAR T cell, TCR T cell, virus specific T cell, NTK cell, tumor infiltrating lymphocyte, hematopoietic stem cell or pluripotent stem cell.

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “TCR” or “T cell receptor” refers to a molecule found on the surface of T cells, or T lymphocytes that is responsible for recognizing fragments of antigen as peptides bound to major histocompatibility complex (MHC) molecules. The TCR is composed of two different protein chains (that is, it is a heterodimer). In 95% of human T cells, the TCR consists of an alpha (α) chain and a beta (β) chain (α/β T cell), whereas in 5% of human T cells, the TCR consists of gamma and delta (γ/δ) chains (γ/δ T cell). The term “TCR T cell” refers to a T cell expressing transgenic TCR.

As used herein, the term “CD3” refers to a T cell co-receptor that helps to activate both the cytotoxic T cell (CD8+ naive T cells) and also T helper cells (CD4+ naive T cells). It consists of a protein complex and is composed of four distinct chains. In mammals, the complex contains a CD3γ chain, a CD3δ chain, and two CD3ε chains.

As used herein, the “TCR/CD3 complex” or “TCR/CD3 complex” is a protein complex involved in the GvHD effect, and consists of variable TCR receptor α and β chains with three dimeric signaling modules CD3δ/ε, CD3γ/ε and CD247ζ/ζ or ζ/η (see FIG. 1). Ionizable residues in the transmembrane domain of each subunit form a polar network of interactions that hold the complex together. Since the cytoplasmic tail of the TCR is extremely short, making it unlikely to participate in signaling, these signaling molecules are vital in propagating the signal from the triggered TCR into the cell. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

The term “DNA gene editing” refers to a type of genetic engineering in which DNA is inserted, deleted, modified or replaced in the genome of a living organism. In 2018, the common methods for such editing use engineered nucleases, or “molecular scissors”. These nucleases create site-specific double-strand breaks at desired locations in the genome. The induced double-strand breaks are repaired through non-homologous end-joining (NHEJ) or homologous recombination (HR), resulting in targeted mutations (‘edits’). As of 2015, four families of engineered nucleases were used: meganucleases, zinc finger nucleases (ZFNs), transcription activator-like effector-based nucleases (TALEN), and the clustered regularly interspaced short palindromic repeats (CRISPR/Cas9) system.

The “CRISPR” was initially described as segments of prokaryotic DNA containing short, repetitive base sequences. In a palindromic repeat, the sequence of nucleotides is the same in both directions. Each repetition is followed by short segments of spacer DNA from previous exposures to foreign DNA (e.g., a virus or plasmid). CRISPR loci typically consist of a clustered set of CRISPR-associated (Cas) genes and the signature CRISPR array-a series of repeat sequences (direct repeats) interspaced by variable sequences(spacers) corresponding to sequences within foreign genetic elements (protospacers). Whereas Cas genes are translated into proteins, most CRISPR arrays are first transcribed as a single RNA before subsequent processing into shorter CRISPR RNAs (crRNAs), which direct the nucleolytic activity of certain Cas enzymes to degrade target nucleic acids.

The term “CRISPR/Cas system” refers to a prokaryotic immune system that confers resistance to foreign genetic elements such as those present within plasmids and phages that provides a form of acquired immunity. Generally, CRISPR/Cas system comprises at least a Cas endonuclease and a guide RNA. The RNA harboring the spacer sequence helps Cas (CRISPR-associated) proteins recognize and cut exogenous DNA.

The term “guide RNA” or “gRNA” generally refers to a RNA molecule, which directs the Cas endonuclease to the target locus and specifically hybridizes to the complementary sequence within the target locus, thereby causing double strand break in the target locus under the action of the endonuclease. gRNA include but not limited to crRNA, sgRNA and other chimeric guide RNAs such as caRNA, csRNA, catRNA. The term “single guide RNA” or “sgRNA” refers to an artificially engineered RNA designed by fusing the crRNA and tracrRNA molecules into a “single-guide RNA” that, when combined with Cas9 protein, could find and cut the DNA target specified by the guide RNA.

In a typical CRISPR system, guide RNA is crRNA, which generally comprises a direct repeat and a spacer sequence. “Direct repeat” refers to repeat sequences interspaced by variable sequences (spacer) within CRISPR locus. “Spacer” refers to viral DNA inserted into a CRISPR locus created from invading viral or plasmid DNA (called “protospacers”). The wild-type Cas9 has a spacer sequence with a length of 20 bp, while the full-length spacer in crRNA of wild type Cpf1 is 24 bp. The crRNA will direct the Cas protein to the invading protospacer sequence on subsequent invasion. But Cas proteins will not cleave the protospacer sequence unless there is an adjacent PAM sequence. The spacer in the bacterial CRISPR loci will not contain a PAM sequence, and will thus not be cut by the nuclease. However, the protospacer in the invading virus or plasmid will contain the PAM sequence, and will thus be cleaved by the Cas endonuclease. For editing genes, guide RNAs are synthesized to perform the function of recognizing gene sequences having a PAM sequence at the 3′-end.

The term “base editing” refers to a new genome editing technology that enables the direct, irreversible conversion of a specific DNA base into another at a targeted genomic locus by utilizing catalytically dead Cas protein (dCas) fused with deaminase enzymes. Importantly, this can be achieved without requiring double-stranded breaks (DSB) in context of DNA. Since many genetic diseases arise from point mutations, this technology has important implications in the study of human health and disease (Landrum, M. J. et al. 2015).

The term “CARs” or “chimeric antigen receptors” refers to engineered receptors which graft an arbitrary specificity onto an immune effector cell (such as a T cell). Typically, these receptors are used to graft the specificity of a monoclonal antibody onto a T cell, with transfer of their coding sequence facilitated by retroviral vectors. The receptors are called chimeric because they are composed of parts from different sources.

The term “CAR T cell” or “chimeric antigen receptor T cell” refers to engineered T cells with chimeric antigen receptors which have predefined specificity towards selected targets. Once encountered with targets, for example cancer cells, CAR T cells destroy the cancer cells through mechanisms such as extensive stimulated cell proliferation, increasing the degree to which the cell is toxic to other living cells i.e. cytotoxicity, and by causing the increased production of factors that are secreted from cells in the immune system that have an effect on other cells in the organism.

The term “central memory T (TCM) cells” refers to T cells expressing CD45RO, C—C chemokine receptor type 7 (CCR7), and L-selectin (CD62L). Central memory T cells also have intermediate to high expression of CD44. This memory subpopulation is commonly found in the lymph nodes and in the peripheral circulation. The TCM cells are thought to contain some properties associated with memory cells stem cells. TCM cells display a capacity for self-renewal due to high levels of phosphorylation of an important transcription factor known as STATS. In mice, TCM cells have been shown to confer superior protection against viruses, bacteria, and cancer in several different model systems compared with terminally differentiated effector cells. It has been reported that in CAR T cells, a younger central memory phenotype is associated with enhanced persistence and a more extensive proliferative capacity. Thus, enhancement of the central memory phenotype would be beneficial for the properties of CAR T cells, especially when said CAR T cells are used for treating diseases.

The term “truncated sgRNA” or “shorter sgRNA” refers to sgRNA with shorter regions of target complementarity <20 nucleotides in length, which can decrease undesired mutagenesis at some off-target sites by 5,000-fold or more without sacrificing on-target genome editing efficiencies. Study shows that the use of shorter or truncated sgRNAs with spacer sequence of 17, 18 or 19 nucleotides of complementarity does not decrease the targeting range of the platform, because target sites with 17, 18 or 19 nucleotides of complementarity will each occur in random DNA with frequencies equal to those with 20 nucleotides of complementarity.

### FIGURES

FIG. 1: An illustration of TCR/CD3 complex structure.

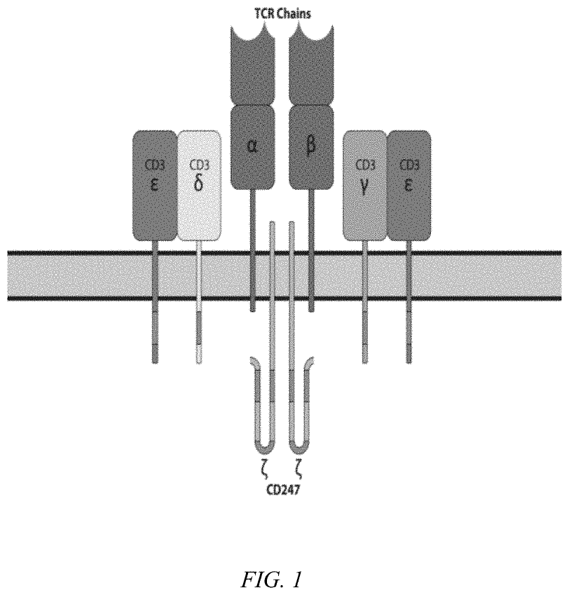

FIG. 2: Disrupting TCR/CD3 complex expression with sgRNAs targeting TCRα chain, CD247ζ chain, CD3ε chain, CD3γ chain and CD3δ chain respectively.

FIG. 3: Central memory phenotype of TCRα chain, CD247ζ chain, CD3ε chain, CD3γ chain and CD3δ chain knockout T cells.

FIG. 4: Cytotoxicity of TCRα chain, CD247ζ chain, CD3ε chain, CD3γ chain knockout T cells. n=3, *P<0.05, by student's t-test.

### EXAMPLES

The following examples are only provided for the purpose of illustration, and will not limit the scope of the present invention in any way.

### Example 1

### TCR/CD3 Complex Disruption in CAR T Cells

Sequences encoding sgRNA targeting the TCRα, CD247ζ, CD3ε, CD3γ and CD3δ gene driven under T7 promoter were cloned into BH-MSG vector respectively.

Cas9 and sgRNA plasmids were linearized before conducting RNA in vitro transcription (IVT). The IVT RNA was stored at −80° C. in nuclease-free vials for single use. Cas9 mRNA was transcribed in vitro using mMESSAGE mMACHINE T7 ULTRA kits (Life Technologies, AM1345, Carlsbad, Calif.). sgRNAs were transcribed using a HiScribe™ T7 High Yield RNA Synthesis Kit (NEB).

To generate modified CAR T cells, primary human CD4 and CD8 T cells were isolated from healthy volunteer donors following leukapheresis by Ficoll-Paque™ PREMIUM (GE healthcare). Then the T cells were first activated by CD3/CD28 beads for 1 day and then transduced with lentivirus expressing a CAR. Then, CAR T cells were washed three times with OPTI-MEM and re-suspended in OPTI-MEM (Invitrogen) at a final concentration of 1-3×10⁸ cells/ml. Subsequently, 20 ug of Cas9 mRNA was electroporated into the cells using a BTX Agile Pulse Max electroporator (Harvard Apparatus BTX) at 360 V and 1 ms on day 3, and various sgRNAs targeting TCRα, CD247ζ, CD3ε, CD3γ and CD3δ gene respectively were electroporated on day 4. Cells were split every 2 days. Cells electroporated with an empty vector were used as control (Mock).

Following electroporation, the cells were immediately placed in 1 ml of pre-warmed culture media and cultured in the presence of IL-2 (300 IU/ml) at 37° C. and 5% CO2. To enrich the cells, CAR T cells washed with Auto MACS buffer were incubated for 15 minutes with CD3 microbeads (Miltenyi Biotec, 130-050-101) at 4° C. After being washed twice, the cells were passed through an LD column (Miltenyi Biotec), and the flow-through fraction was collected for further use.

The spacer sequences of sgRNAs used in the example were selected from Table 1 below.

TABLE 1
The spacer sequences of sgRNAs
targeting the TCRα, CD247ζ,
CD3ε, CD3γ and CD3δ gene.

| Name | Spacer Sequence |
|---|---|
| CD247ζ-sgRNA-1 | GTGGAAGGCGCTTTTCACCG |
| | (SEQ ID NO: 1) |
| CD247ζ-sgRNA-2 | AAAGGACAAGATGAAGTGGA |
| | (SEQ ID NO: 2) |
| CD247ζ-sgRNA-3 | TTTCACCGCGGCCATCCTGC |
| | (SEQ ID NO: 3) |
| CD247ζ-sgRNA-4 | CAGGCACAGTTGCCGATTAC |
| | (SEQ ID NO: 4) |
| CD247ζ-sgRNA-5 | GATGGAATCCTCTTCATCTA |
| | (SEQ ID NO: 5) |
| CD247ζ-sgRNA-6 | GACGCCCCCGCGTACCAGCA |
| | (SEQ ID NO: 6) |
| CD247ζ-sgRNA-7 | GGCACAGTTGCCGATTACAG |
| | (SEQ ID NO: 7) |
| CD3ε-sgRNA-1 | GGGCACTCACTGGAGAGTTC |
| | (SEQ ID NO: 8) |
| CD3ε-sgRNA-2 | GGCCTCTGCCTCTTATCAGT |
| | (SEQ ID NO: 9) |
| CD3ε-sgRNA-3 | GCCTCTTATCAGTTGGCGTT |
| | (SEQ ID NO: 10) |
| CD3ε-sgRNA-4 | GATGAGGATGATAAAAACAT |
| | (SEQ ID NO: 11) |
| CD3ε-sgRNA-5 | GAACTTTTATCTCTACCTGA |
| | (SEQ ID NO: 12) |
| CD3ε-sgRNA-6 | CCATATAAAGTCTCCATCTC |
| | (SEQ ID NO: 13) |
| CD3ε-sgRNA-7 | TTGACATGCCCTCAGTATCC |
| | (SEQ ID NO: 14) |
| CD3ε-sgRNA-8 | GGGGCTTCGCGGTAAAGTTG |
| | (SEQ ID NO: 15) |
| CD3ε-sgRNA-9 | GGGGGGCTTCGCGGTAAAGT |
| | (SEQ ID NO: 16) |
| CD3ε-sgRNA-10 | GGCTGGTGGGTTGCCCCCAA |
| | (SEQ ID NO: 17) |
| CD3ε-sgRNA-11 | GGGCCCCAGGCCGGCGGTGA |
| | (SEQ ID NO: 18) |
| CD3γ-sgRNA-1 | ACTTTGGCCCAGTCAATCAA |
| | (SEQ ID NO: 19) |
| CD3γ-sgRNA-2 | GTGTATGACTATCAAGAAGA |
| | (SEQ ID NO: 20) |
| CD3γ-sgRNA-3 | TGACTATCAAGAAGATGGTT |
| | (SEQ ID NO: 21) |
| CD3γ-sgRNA-4 | AATATCACATGGTTTAAAGA |
| | (SEQ ID NO: 22) |
| CD3γ-sgRNA-5 | TTTAAAGATGGGAAGATGAT |
| | (SEQ ID NO: 23) |
| CD3γ-sgRNA-6 | GAATCTGGGAAGTAATGCCA |
| | (SEQ ID NO: 24) |
| CD3γ-sgRNA-7 | AGTCATACACCTTAACCAAG |
| | (SEQ ID NO: 25) |
| CD3γ-sgRNA-8 | GGTGGAGTTCGCCAGTCGAG |
| | (SEQ ID NO: 26) |
| CD3γ-sgRNA-9 | GGAATGCCAAGGACCCTCGA |
| | (SEQ ID NO: 27) |
| CD3γ-sgRNA-10 | GGACTATCAAGAAGATGGTT |
| | (SEQ ID NO: 28) |
| CD3γ-sgRNA-11 | GGCAGTTCTGACACACTGTA |
| | (SEQ ID NO: 29) |
| CD3δ-sgRNA-1 | GAACATAGCACGTTTCTCTC |
| | (SEQ ID NO: 30) |
| CD3δ-sgRNA-2 | GTTTCTCTCTGGCCTGGTAC |
| | (SEQ ID NO: 31) |
| CD3δ-sgRNA-3 | GATACCTATAGAGGAACTTG |
| | (SEQ ID NO: 32) |
| CD3δ-sgRNA-4 | GCTCTCAGACATTACAAGAC |
| | (SEQ ID NO: 33) |
| CD3δ-sgRNA-5 | GGACCTGGGAAAACGCATCC |
| | (SEQ ID NO: 34) |
| CD3δ-sgRNA-6 | GGTCCAGGATGCGTTTTCCC |
| | (SEQ ID NO: 35) |
| CD3δ-sgRNA-7 | TTCCTCTATAGGTATCTTGA |
| | (SEQ ID NO: 36) |
| CD3δ-sgRNA-8 | GGCGTCGTAGGTGTCCTTGG |
| | (SEQ ID NO: 37) |
| CD3δ-sgRNA-9 | GGATGGGGGGAAAGCCGGTA |
| | (SEQ ID NO: 38) |
| CD3δ-sgRNA-10 | GAAGGACAAGATGAAGTGGA |
| | (SEQ ID NO: 39) |
| CD3δ-sgRNA-11 | GGGTAGGGCCGACGTGTCGA |
| | (SEQ ID NO: 40) |
| TCRα-sgRNA-1 | AGAGTCTCTCAGCTGGTACA |
| | (SEQ ID NO: 41) |

### Example 2.

### TCR/CD3 Complex Expression in Modified CAR T Cells

It has been reported that disrupting components of TCR (α or β chain) in α/β T cell can lead to the abolishment of TCR/CD3 complex expression, thereby prevent the GvHD effect of allogeneic T cells. We assume that the integrity of TCR/CD3 complex require the fully assembly of TCR components, CD3 components and CD247ζ.

Thus, to test whether CD3 components and CD247ζ disruption could lead to TCR/CD3 complex disruption, we knocked out the CD3 components, including CD3γ, CD3δ, and CD3ε as well as CD247ζ with CRISPR gene editing.

Modified CAR T cells were obtained according to the method described in Example 1. Genomic DNA of modified CAR T cells were extracted, Sanger sequencing of PCR products flanking the targeted sites were performed to confirm the targeted editing at DNA strands. Results were also analyzed by TIDE (Tracking of Indels by DEcomposition) software. Genomic disruption and insertion in the TCRα, CD247ζ, CD3ε, CD3γ and CD3δ gene were confirmed (data not shown).

Next, TCR/CD3 expression was measured by flow cytometry by staining with APC-anti-CD3 antibody (Cat No. 555335, BD Biosciences).

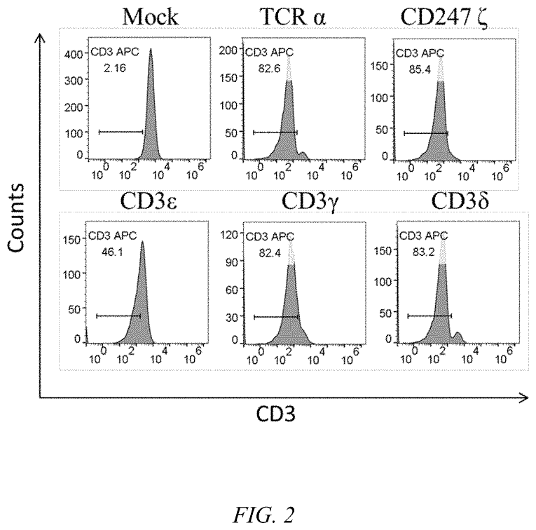

FIG. 2 shows the results of flow cytometry, wherein the TCR/CD3 expression was expressed as number of CD3 negative cells. As shown in FIG. 2, there's almost no CD3 negative cell population in Mock electroporated group, but efficient gene ablation was observed with CRISPR targeting TCR α chain, different CD3 components and CD247ζ. These results demonstrated that knockout of CD3 components and CD247ζ could also abolish TCR/CD3 complex expression in T cells.

### Example 3.

### Determination of the Phenotype of Modified CAR T Cells

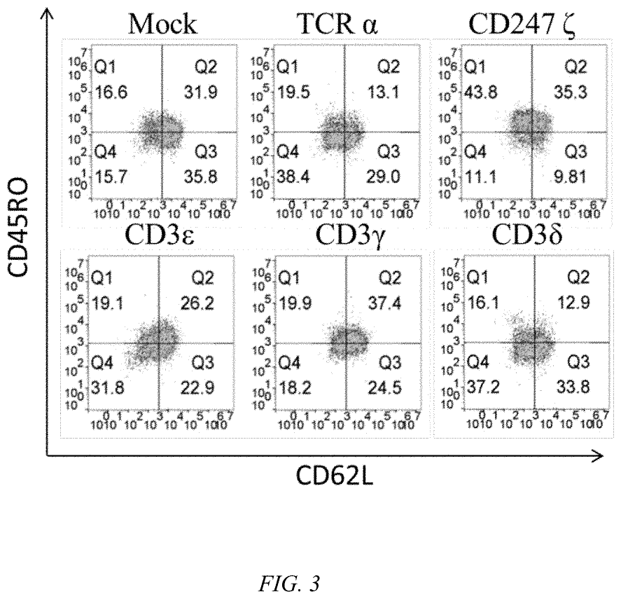

To measure the central memory phenotype of CD3 component and CD247ζ disrupted T cells, CD45RO and CD62L expressions were determined by flow cytometry. The results are shown in FIG. 3.

Surprisingly, we found that CD247ζ, CD3ε and CD3γ knockout CAR T cells exhibited more CD45RO and CD62L double positive central memory phenotype than TCRα knockout CAR T cells. Specifically, there are around 31.9% CD45RO and CD62L double positive central memory cells within Mock T cell population. The CD45RO and CD62L double positive cell population decreased to 13.1% within TCR α knockout cells. However, CD247ζ, CD3ε and CD3γ disrupted T cells contain higher number of CD45RO and CD62L double positive central memory cells than TCRα knockout, at a level equivalent to Mock T cells. It was also noted that CD3δ knockout results in a comparable central memory phenotype as TCRα.

It is known that a younger central memory phenotype of CAR T cells is associated with enhanced persistence and a more extensive proliferative capacity, thereby would be beneficial for prolonged capacity of CAR T cells. Thus, this data indicates that CD247ζ, CD3ε and CD3γ disruption are more favorable for potent universal CAR T cell generation than TCRα disruption.

### Example 4.

### Determination of the Cytotoxicity of Modified CAR T Cells

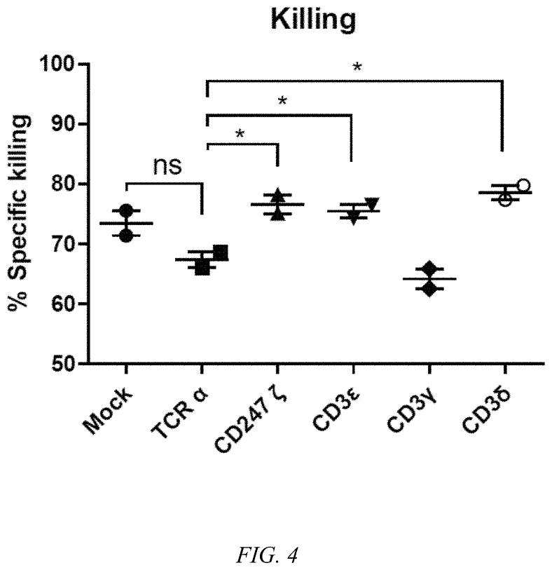

To test whether TCR/CD3 disruption would affect the effector function of the CAR T cells, co-culture of modified CAR T cells with target Nalm6 cells were performed. Specifically, the cytotoxicity of modified CAR T cells was tested by a modified version of luciferase-based CTL assay, wherein Nalm6 tumor cells were generated and employed. The resulting Nalm6 cells were re-suspended at 1×10⁵ cells/mL in R10 medium and incubated with modified CAR T cells overnight at 37° C. Then, 100 μL of the mixture was transferred to a 96-well black luminometer plate. Next, 100 μL of substrate was added, and the luminescence was immediately determined. The results are shown in FIG. 4.

It was surprisingly found that the modified CAR T cells exhibit prominent difference in target specific killing. In particular, although there is no significant difference between Mock and TCRα chain knockout CAR T cells, CD247ζ, CD3ε and CD3δ knockout CAR T cells showed significantly higher in vitro lytic capacity, while CD3γ knockout CAR T cells showed killing capacity comparable to that of TCRα knockout CAR T cells.

Taken together, all these data suggest that, CD3γ, CD3δ, and CD3ε as well as CD247ζ disruption results in equally efficient TCR/CD3 complex ablation as TCR components (TCR α chain or β chain) disruption, while showing higher central memory phenotype and enhanced target tumor killing capability.

1. A modified T cell, wherein the expression level of TCR/CD3 complex is disrupted or reduced by repressing or abolishing the expression of at least one gene selected from CD3γ, CD3δ, and CD3εand CD247ζ.

2. The modified T cell of claim 1, wherein the modified T cell further exhibits repressed or abolished expression in TCR α and/or β gene.

3. The modified T cell of claim 1, wherein the T cell is a T cell, CAR T cell, TCR T cell, virus specific T cell, NTK cell, tumor infiltrating lymphocyte, hematopoietic stem cell or pluripotent stem cell.

4. A pharmaceutical composition comprising the modified T cell of claim 1.

5. Use of the modified T cell of claim 1 in the manufacture of a medicament for treating or preventing cancer, infections or autoimmune diseases.

6. A method of enhancing the central memory phenotype of a T cell, comprising disrupting the expression level of TCR/CD3 complex by repressing or abolishing the expression of at least one gene selected from CD3γ, CD3δ, and CD3εand CD247ζ in said T cell.

7. A method of enhancing the tumor killing capability of a T cell, comprising disrupting the expression level of TCR/CD3 complex by repressing or abolishing the expression of at least one gene selected from CD3γ, CD3δ, and CD3εand CD247ζ in said T cell.

8. A method of abolishing the GvHD effect of a T cell, comprising disrupting the expression level of TCR/CD3 complex by repressing or abolishing the expression of at least one gene selected from CD3γ, CD3δ, and CD3εand CD247ζ in said T cell.

9. The method according to claim 6, further comprises repressing or abolishing the expression of TCR α and/or β gene.

10. The method according to claim 6, wherein disrupting the expression level of TCR/CD3 complex is achieved by gene mutation, RNA-mediated inhibition, RNA editing, DNA gene editing or base editing.

11. The method according to claim 10, wherein the gene editing method involves the use of a nuclease selected from a meganuclease, ZFN, TALEN, and Cas enzyme.

12. The method according to claim 11, wherein the nuclease is a Cas9 enzyme.

13. The method according to claim 11, wherein the Cas enzyme is used in a CRISPR/Cas system comprising at least one sgRNA comprising a spacer sequence selected from SEQ ID NO: 1-40, or a truncated spacer sequence with at least 17 nucleotides identical or complementary to a spacer sequence selected from SEQ ID NO: 1-40.

14. A T cell obtained according to the method of claim 6.

15. A CRISPR/Cas system comprising at least one sgRNA comprising a spacer sequence selected from SEQ ID NO: 1-40, or a truncated spacer sequence with at least 17 nucleotides identical or complementary to a spacer sequence selected from SEQ ID NO: 1-40, wherein expression of said CRISPR/Cas system in a T cell results in disruption of TCR/CD3 complex expression in said T cell.

16. A construct comprising a polynucleotide encoding a RNA molecule that is essentially identical or essentially complementary to a transcript sequence of at least one gene selected from CD3γ, CD3δ, and CD3εand CD247ζ or fragments thereof, wherein the expression of the construct in a T cell results in disruption of TCR/CD3 complex expression in said T cell.

17. The construct according to claim 16, wherein the RNA molecule is an antisense RNA, a miRNA, a siRNA or a long non-coding RNA.
